(19) United States
(12) Patent Application Publication
(10) Pub. No.: US 2008/0210225 A1
(43) Pub. Date: Sep. 4, 2008

(54) DISPOSABLE ANTISTATIC SPACER

(75) Inventor: THOMAS GEIGER, Philadelphia, PA (US)

Correspondence Address: Thomas Geiger, 7731 Walker St., Philadelphia, PA, 19136, US

(73) Assignee: RAPHA INSTITUTE FOR HEALTH, Philadelphia, PA (US)

(21) Appl. No.: 12/034,821

(22) Filed: Feb. 21, 2008

Related U.S. Application Data
(60) Provisional application No. 60/904,087, filed on Mar. 1, 2007.

(51) Int. Cl. A61M 11/00

(52) U.S. Cl. 128/200.14

(57) ABSTRACT

A “disposable antistatic spacer” is presented for use as a universal medicinal inhalant applicator offering an inexpensive tube configuration with antibacterial and biodegradable characteristics for efficient administration of pharmaceuticals; including antibiotics, vaccines and brochodilators; having removable parts for adaptability to diverse pressurized inhalant pumps and is furthermore; an interchangeable platform for other pulmonary therapeutic devices.

### FIELD OF INVENTION

“This invention relates to the field of medicine, specifically to an improved spacer tube accessory device for administering different pulmonary therapeutics”.

### BACKGROUND OF THE INVENTION

As early as the 1890's the “First Generation” of devices to help facilitate medicine inhalation first appeared. Hoell's “Breathing Apparatus” U.S. Pat. No. 506,368 Oct. 10, 1893 listed a glass cylinder, tubular extension, drum portion and a spray chamber as individual components.

Today, inhalant devices are less complex, smaller and more efficient, yet they still offer cylindrical, tubular, chamber and drum like components within their apparatus. Currently, a drum canister called the Pressurized Metered Dose Inhaler (pMDI) is the most widely used drug-propelling product in the world for treating respiratory dysfunctions.

Conveniently pocket-sized, pMDI's utilize a gas propellant to expel the medicine from the inhaler. Introduced in the 1950's, pMDI's revolutionized treatment of Asthma and COPD because of their ease of use and rapid delivery to the intended site of action, the large blood supply of the lung. Virtually the entire cardiac output pumps through the alveolar capillaries, which makes inhaled delivery to the alveoli an attractive option for the systemic delivery of drugs.

Accordingly, vaporized therapy is quickly moving from a modality aimed solely at relieving lung diseases, to one used for administering different types of drugs. One trial (Beth L. Laube, PhD Chest 114,1734-1739) has shown that inhaled insulin could effectively control blood glucose levels. Because this along with other investigations has revealed promising outcomes, a number of other drugs including vaccines are now undergoing preclinical inhalation testing.

However, the pMDI is very inefficient. The efficacy of inhalers depends on their ability to deliver sufficient medicine to the lower respiratory tract. But with pMDI's the majority of drug impacts in the oropharynx (upper airway) and only a scant 15-20% of the drug dose reaches the alveoli (lower airway).

This results in a majority of the drug being wasted and as a consequence, patients will constantly actuate more puffs from their pMDI's just to obtain relief. This regrettably contributes to overdosing and brings unwanted systemic side effects like tachycardia. To overcome this problem various approaches have been attempted to help facilitate better inhaler administration.

Numerous pMDI accessory tubes, cylinder drums and air chambers have been developed to overcome this drug delivery deficiency. By essentially slowing down (spacing) the aerosol dispersion between the patient and the pMDI this “Second Generation” spacer creates a more natural respirable flow. By maneuvering medicine past the upper airways spacers help pMDI's deliver more medicine to the lungs. Unfortunately they are not widely accepted because they suffer from one or more serious inadequacies:

(a) Their average cost is around twenty dollars. Which is economically limiting for most patients who can just barely afford to pay for their medicines.

(b) Their confusing. Farmer's apparatus U.S. Pat. No. 6,494,202 offers technology that includes a full mask, an expandable spring-loaded reservoir and an adjustable exhalation valve, but its beyond the average patients understanding and ability to operate correctly.

(c) Their maintenance requirements. Each inhaled and exhaled warm breath offer bacteria the perfect environment. The “Aerochamber” by the Monaghan Corporation comes with valves, whistles and available add-on accessories that offer many challenges for keeping viral microorganisms off. The exploded detail in U.S. Pat. No. 5,816,240 by Komesaroff reveals how daunting the task of spacer maintenance and cleaning is for the average patient.

(d) Their disposability is limited. U.S. Pat. No. 4,953,545 issued Sep. 4, 1990 to McCarty for a “Disposable Respiratory Medication Dispersion Chamber” is a spacer that cannot be discarded in part while sustaining the other parts for additional service thereby conserving resources.

(e) Their manufacture excludes two basic elements needed to perform effectively. Many publications including CHEST 1998:114:1676-1680 by Finley and Zuberbuhler pg. 1676 paragraph 2 “. . . it is desirable to use holding chambers with valves that prevent rebreathing of the exhaled air, otherwise little drug will be inhaled from the holding chamber” reveals the first important element as a one-way valve, which is a must have trend for spacers that specifically helps uncoordinated patients. The second needed element that is missing in McCarty's cited above and Sladek's U.S. Pat. No. 6,679,252 B2 is a chamber adequately spaced to provide a proper respirable flow. Sufficient spacing allows for time by distance to slow the propelled dispersion for better aerosol delivery. Moreover, several “Second Generation” spacers including U.S. Pat. No. 5,477,849 issued Dec. 26, 1995 to Fry, fail to include either of these two much-needed elements.

(f) Their electrostatic properties. Static cling is an inherent problem of the synthetic polymers used in their walls or in parts of their chambers. Most “Second Generation” spacers are plastic and electrostatic charge can build up on their surface walls. Compelling evidence collected shows the consequence of electrostatic charge on plastic devices to aerosol drug retention within these devices, resulting in significant reduction of the medicine available for inhalation. They demonstrate a triboelectric effect, which is a type of contact electrification in which certain materials become electrically charged after they come into contact such as through rubbing with other material. “Historically, respiratory drug delivery has disregarded the subject of electrostatics” writes Professor Pert in “Relevance of Electrostatics in Respiratory Drug Delivery” and concludes: “that spacer electrostatic charge does influence drug retention in spacer devices and it would therefore seem obvious that manufacturers of spacer devices should use only non-electrostatic materials in the future”.

The current alternatives are limited to the premium non-static metal spacers like the “Vortex” by the Pari Company or the expensive anti-static polymer types like the “Aerochamber Max” by Monahan Medical Corp. But their price is even more cost prohibitive to most patients. Clearly, these “Second Generation” spacers have not explored materials having antistatic attributes that are less expensive to produce.

(g) Their lack of biodegradability. Sladek's spacer has integral valves and a window made of plastic, which along with all plastic “Second Generation” spacers require labor-intensive dismantling for safer ecological disposal. Furthermore, the chemical additives like Stat-Ban® custom anti-static polymer used in the Aerochamber Max that give plastic products desirable performance properties can have negative environmental and human health effects. These effects include direct toxicity, as in the cases of lead, cadmium, and mercury; or carcinogens, as in the case of diethyl hexylphosphate (DEHP). Problem chemicals are used as plasticizers, colorants, heat stabilizers, and barrier resins. A single resin type might be mixed with many such additives, adding complexity to the chemical composition, possibly generating new classes of incompatible resins creating a recycling problem because blow-mold resin grades and injection-mold grades must be separated for primary recycling applications. People are exposed to these chemicals not only during manufacturing, but also by using plastic products. Because some chemicals migrate out from the polymer a migration potential exists for traces of monomers, oligomers, stabilizers, “anti-static” nucleating agents, and reaction products of the polymer or its additives. Such substances may be toxic. It is readily apparent that this generation of spacers has not investigated renewable, environmentally friendly embodiments that are easier and safer to process for recycling.

(h) Their lack of adaptability. Because of their set embodiments they cannot be transformed in place, or equipped by inexpensive modification for use by different pulmonary apparatus. A CMAJ Asthma Consensus “Inhalation Devices and Propellants” Nov. 30, 1999 page 46 paragraph 2 points out

“Because the efficacy and side effects of inhaled medications are highly dependent on the device, drug and user, the response to a drug delivered from a specific inhaler may differ from the response to different drugs of the same class delivered by the same-inhaler or the same drug delivered by a different device”. Not having a platform device that is adaptable to different pressurized medicinal applicators or even changeable to other pulmonary devices such as peak flow meters is problematic with the present generation of devices.

(i) Their lack of a replaceable valved mouthpiece. Which would offer the advantage of one chamber being used repeatedly and safely with different patients. This benefit of replacing the valved mouthpiece while the chamber remains would be economically relevant especially for mass aerosol inoculation campaigns.

For these reasons among others, there remains a need for an innovative “Next Generation” spacer that can be used conveniently to: administer propelled inhalant medicines of all types without degrading their properties or dosage, is inexpensive to manufacture, can be supplied in part; in whole or en masse, can be used repeatedly or disposed of after one use, is easier to use and to maintain, incorporates features like a replaceable mouthpiece, adaptable end caps, is adequate in size, comes with a one way valve, offers convertibility to different inhalant devices, is durable and crush resistant, has antistatic and biodegradable characteristics which is easier to recycle and meets the need for an inexpensive “Next Generation” spacer that is more environmentally sustainable and is more readily acceptable by the public.

### SUMMARY

The present invention, a “Next Generation” pMDI spacer generally comprises a tube body, a staged mouthpiece, a one-way valve and an adaptable inlet end orifice. The single tube body is made of a moisture and crush resistant material that is inexpensive, readily available and will not induce a static electrical charge. This novel device is affordable, sustainable, biodegradable and safer to recycle as well. Accordingly, several objects and advantages of the original invention include; an inexpensive, revolutionary, easy to use “Disposable Antistatic Spacer” that better facilitates dispensing of inhalant medications, vaccines and dry powdered medicants more effectively, is a safer device that offers the modern antibacterial benefit of disposability after one or numerous applications, is an innovative platform for other pulmonary therapeutic devices, and notably provides an efficient disposable valved mouthpiece and an economical tube cartridge spacer desirable for needle-free mass aerosol inoculations.

### OBJECTS OF THE INVENTION

Accordingly, some potential advantages and objects of the present invention are:

1. To provide a practical spacer with a lower cost that would allow patients to obtain three spacers: one for their home, one for their automobile and one for their workplace, at a price costing less then one current spacer device.

2. To provide a novel spacer with a less complex design, that is simpler and easier to use without complicated instructions.

3. To provide a unique spacer that is maintenance free with no assembly or cleaning required.

4. To provide an original spacer with disposability convenience, whereas the whole spacer or just parts of the spacer can be disposed of as an anti-bacterial disposal benefit.

5. To provide an effective spacer with the basic elements needed for the most efficient drug delivery.

6. To provide an inspired spacer with dielectric anti-static properties made from inexpensive natural materials.

7. To provide a new spacer made from renewable, recyclable resources, that offers biodegradable properties, which are sustainable and easier to recycle.

8. To provide a pioneering spacer with an adaptable platform for different inhalant medicine-propelling devices and is interchangeable for other pulmonary therapeutic devices.

9. To provide an innovative spacer with a replaceable valved mouthpiece and tube cartridge for mass needle free inoculation campaigns.

Further advantages are to provide a modifiable spacer transition between the patient and the medicinal applicator, offering a convertibility aspect that is readily transformable in place or can be inexpensively customized for different types of inhalant applicators. Another advantage of the present invention is its break through platform technology. This pioneering design offers economical manufacturing capabilities for other disposable pulmonary therapeutic devices. Some examples include; a breathing flow exerciser, a respiratory muscle trainer, a sputum cough inducer and a peak flow meter. Public and private consumers will benefit from a much-needed lower costing innovative pulmonary therapeutic platform device that is conveniently disposable.

Another important aspect of the present invention is to provide a new “Next Generation” pressurized medicine delivery device that has novel features that are popular, affordable, relevant, environmentally responsible and not offered by any other. And additionally answers the global need for a low cost needle free device that delivers mass aerosol vaccines efficiently and safely. Thus, resulting in a new and novel drug delivery device that is not apparent, obvious, or suggested either directly or indirectly by any other.

At least one of the preceding objects is met in whole or in part by the present invention. In reference to such, there is to be a clear understanding that the present invention is not limited to any method, material or detail of construction, fabrication, application of use that has been described and illustrated herein. Accordingly, any variation of fabrication, use, material, or application is to be considered as apparent and as an alternative embodiment of the present invention.

### DRAWINGS—FIGURES

In the drawings, closely related figures have the same number but different alphabetic suffixes.

FIG. 1 shows an inner sectional aspect of the disposable tube spacer.

FIG. 2 shows an exploded view of the tube spacer.

FIGS. 3a to 3c shows various combinations and views of the mouthpiece.

FIG. 4 shows a valve diaphragm.

FIG. 5 shows a valve seat with valve stem.

FIG. 6 shows the valve diaphragm attached to valve seat by valve stem.

FIGS. 7a to 7b show horizontal views of the valve seat having various combinations of valve stem heads.

FIGS. 8a to 8d show end caps with various combinations of inlet apertures.

FIG. 9 shows a tube body with end cap.

FIG. 10 shows a tube body with mouthpiece.

FIG. 11 shows a cutaway view of tube body substrate.

FIG. 12 shows a disposable inoculation cartridge and valved mouthpiece with a mass inoculator pump (M.I.P.).

### DRAWINGS & REFERENCE NUMERALS

14 mouthpiece 16 mouthpiece lip stage
18 mouthpiece valve stage 20 mouthpiece body stage
22 tube body 24 end cap
26 end cap inlet 28 valve seat stem
30 valve diaphragm 32 valve seat
34 custom mouthpiece lip stage 36 mouthpiece inner lip stage
38 mouthpiece inner valve stage 40 mouthpiece inner body stage
42 valve diaphragm aperture 44 valve seat passageway
46 valve seat cross member 48 mass inoculator pump
50 inoculation cartridge 52 outer layer
56 core layer 60 anti-static layer

### DETAILED DESCRIPTION

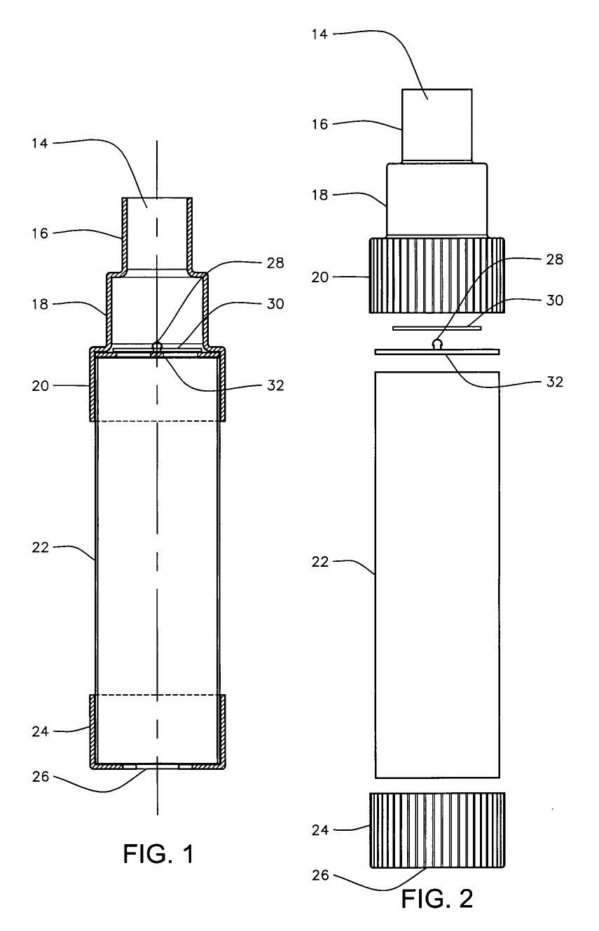

An embodiment of the spacer of the present invention is illustrated in FIG. 1 inner sectional view and FIG. 2 exploded view. The spacer generally has a thin chamber or tube body 22 of uniform cross section consisting of a laminated spiral wound non-electrostatic substrate material, which does not promote condensation or attraction and offers excellent dielectric strength. The tube body 22 as a whole should combine to promote its antistatic properties in the preferred embodiment, but should at least have an antistatic layer laminated on the inner tube body 22 core. This laminate is nearly neutral on the triboelectric series (see below) having a small positive charge tending not to attract or give up a charge and is laminated uniformly for complete protection.

This embodiment considers for the antistatic layer a sustainable white paper liner available from the Wausau-Mosinee Paper Co. having generally a thickness about 0.127 mm and a dielectric strength rated to 1200 at 240 volts/mm. Yet, other types of antistatic layers can be applied like rag paper, cotton composite paper or any commercially available organic or inorganic antistatic paper like VPCI-145, which is a natural antistatic paper from Cortec Corporation St. Paul, Minn. that eliminates static electricity buildup through the use of an environmentally friendly coating made from soybean oil coated onto the surface of the paper. The layer could also be an antistatic paper close to the examples found in Yasuda's U.S. Pat. No. 3,881,988 May 6, 1975 choosing the organic natural (clay) antistatics would be better environmentally but any antistatic coating or spray could be investigated for the tube body 22 inner layer but should try to be biodegradable and easy to recycle.

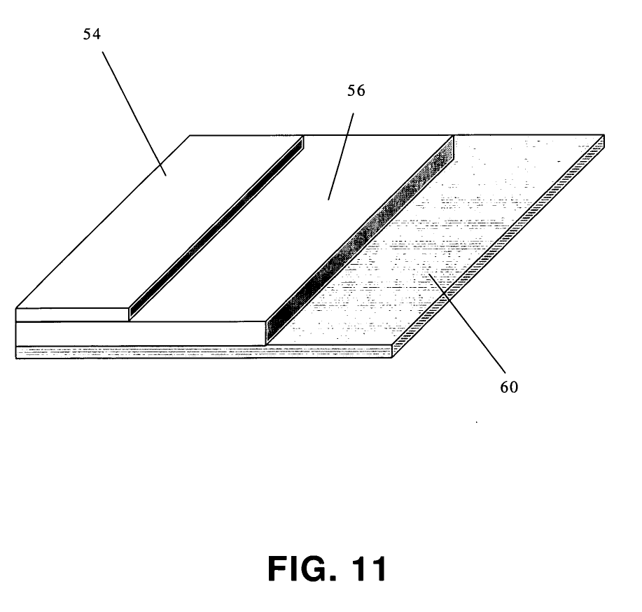

The preferred cover layer 54 FIG. 11 of the present tube body 22 is an environmentally friendly gloss white wrap from the Wausau-Mosinee Paper Co. generally 0.127 mm thickness and helps protect against static buildup, still other sizes and materials including; plain or color or plastic or printed or sprayed or dipped or even natural Kraft from other manufactures would also work.

A natural 100% biodegradable Kraft paper of about 0.030″ thickness is considered for the current tube body 22 inner core. But, the tube body 22 can also consist of any color and any material that can be convoluted or spiral wound such as: tissue, chipboard, fiberboard, textile, vinyl, nylon, rubber, leather, polyethylene or polypropylene fibers, various impregnated or fibrous materials, plasticized or composite materials, cardboard or various paper products but should offer little or no static electrical charging characteristics. Nonetheless, the core surfaces do not have to be laminated but can standalone, or can utilize coatings such as fabric, wax, antistatics, powder, film, oil, silicone, ink or paint. The inner core may also be of a solid material or blow foam product that is laminated between two sheets or may even be fabricated of a corrugated cardboard type.

One preferred shape of the tube body 22 is a straight cylindrical form with a hollow center but it can be oblong, rectangular, triangular or even cone-shaped. The tube body wall can average typically from 0.5 mm to 2.0 mm in ply thickness with overall inner dimension roughly from 20 mm to 70 mm and of a length varying from 80 mm to 200 mm still other sizes may be explored for performance.

At one end of the present embodiment exploded view FIG. 2 is a mouthpiece 14 with three stages featuring an outlet opening at the pinnacle of the lip or first stage 16 that features rounded or beveled edges for patient comfort transitioning to a second stage or mouthpiece valve stage 18 that allows space for free flow valve flap movement and then further transitions to a third stage or mouthpiece body stage 20. This final stage includes striations FIG. 2 or gripping grooves pictured on the outer surface of the mouthpiece body stage 20 and pictured on the end cap 24 to allow for better handling and provides anti-roll control. The mouthpiece 14 may also be configured into a two stage or even a one-stage design but all stages can be with or without an integral one-way valve assembly exploded view FIG. 2 that consists of a valve seat stem 28 a valve diaphragm 30 and a valve seat 32 note the term “valve assembly” used hereafter will refer to all valving components: valve seat 32 valve seat stem 28 and valve diaphragm 30 combined or to other combinations know to the art.

Important to this design is that other valve assemblies like a duckbill flap, or multiple split center flap or any other usable valve configuration including a two way valve set up for inhalation and exhalation may also be evaluated.

Located under the base of mouthpiece 14 in FIG. 2 under the bottom of the mouthpiece body stage 20 is an opening relative in size for receiving the tube body 22 and is shown seated in the cutaway view of FIG. 1. Also in FIG. 1 is a cutaway view revealing the valve assembly and shows the valve seat 32 sandwiched interially between the tube body 22 and the inside of the mouthpiece body stage 20 of the mouthpiece 14.

FIGS. 7a to 7c shows examples of the valve seat stem 28 with different mushroom, arrow or cross cap valve stem heads that could be used to hold the diaphragm 30 in place, of course other valve stem or stems may be considered applicable as well. Typically the diaphragm 30 of FIG. 4 should be made with a material that retains its shape after flexing like latex, but silicone, plastic, waxed paper or other composite substances can be utilized.

A wagon wheel design of the inventions valve seat 32 is depicted in FIG. 5 revealing four apertures or valve seat passageway 44 for flow through when the valve diaphragm 30 is open. FIG. 6 outlines the diaphragm 30 resting on the valve seat 32 and held in place by valve stem 28 with the mushroom head. It is important to understand that other valves and valve attachments or stems along with different valve seats can work and can be tested.

In FIG. 6 the valve seat passageway 44 is pie shaped and completely covered when diaphragm 30 is at rest. The valve diaphragm 30 of FIG. 4 introduces a hole or valve diaphragm aperture 42 that allows the valve seat stem 28 head to come through. The valve diaphragm aperture 42 should be sized a little smaller then the total width of the valve seat stem 28 head to keep the valve diaphragm 30 below in place on the valve seat stem 28 which should be about the same size or a bit wider then the valve seat stem 28.

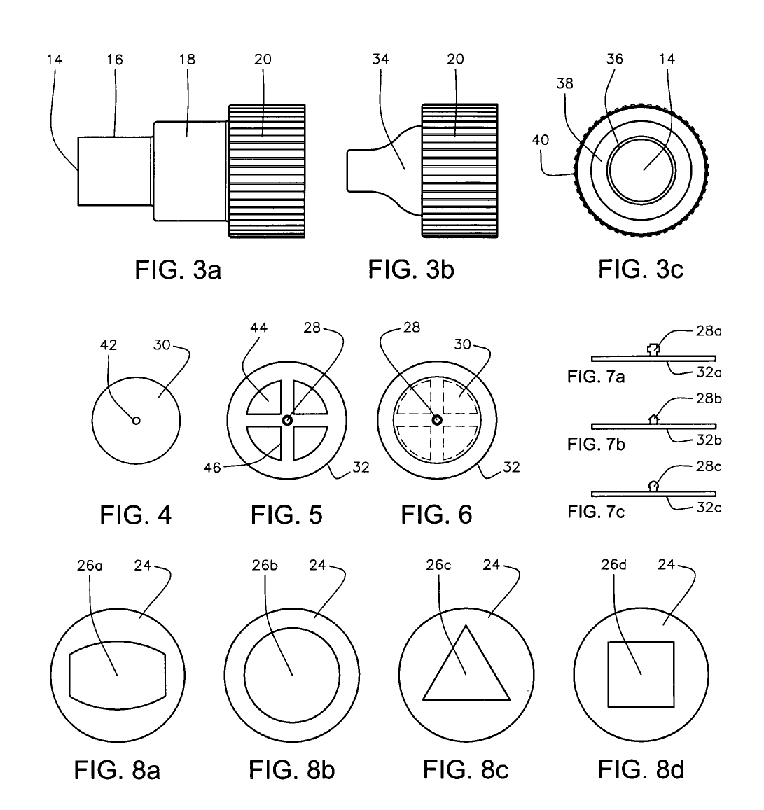

Illustrated in FIG. 3a is a three-stage valved mouthpiece 14. This houses the valve assembly in the body stage 20 a valve opening space in the mouthpiece valve stage 18 and a mouth rest located on the mouthpiece lip stage 16. It is to be understood hereafter that “mouthpiece” can refer to any stage combination and furthermore refers to coming equipped integrally with or without a valve assembly. Another mouthpiece in FIG. 3b depicts a two-stage configuration featuring a custom mouthpiece lip stage 34 and has a mouthpiece body stage 20.

Not pictured, yet understand, that a one-stage or one-piece mouthpiece configuration could work and should also be considered with or without a valve assembly. The mouthpiece may be fabricated from paper, cardboard, plastic or any composite that is formable and relevant to this medical device. An interior view FIG. 3c of mouthpiece 14 illustrates the inside wall and inner body stage 40 ledge portion of the third stage where the valve seat 32 butts up against, a mouthpiece inner valve stage 38 of the second stage which gives room for the valve to open, and an interior wall 36 of the mouthpiece lip stage 16.

On the opposite end of the tube body 22 in FIG. 2 is an end cap 24 with similar outer wall striations as the mouthpiece body stage 20. An end cap inlet 26 of FIGS. 8a to 8d page 2/3 depicts the examples of custom inlet openings that can be configured for the bottom of the end cap 24. The end cap 24 is replaceable with different easily manufactured end cap inlet 26 configurations for receiving different pressurized inhaler devices. Respective in size to the base of mouthpiece 14 and located under end cap 24 is an opening relative in size for receiving tube body 22. Equally, the mouthpiece 14 and end cap 24 should receive the tube body 22 inserted generally between 10 mm to 25 mm deep.

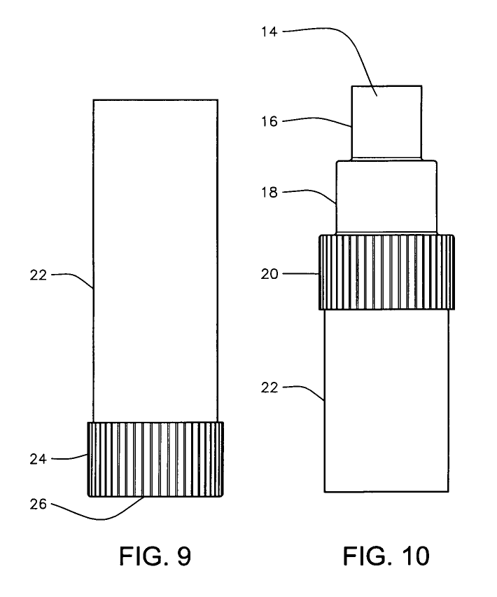

Additional embodiment examples shown in FIGS. 9 and 10 show different sizing and modifications contemplated for the present invention. In FIG. 9 the disposable tube body 22 without mouthpiece 14 is prepared to receive medications through inlet 26 that can be inhaled at the opposite end either nasally or with an added facemask (not shown).

The proposed embodiment in FIG. 10 of the disposable tube body 22 is modified shorter shown without an end cap 24 but featuring a three-stage mouthpiece 14 with a one-way valve assembly ready for use with different inhalant pumps.

A cutaway view of the laminates or layers of the tube body 22 material in FIG. 11 reveals a cover layer 54 a core layer 56 and an antistatic layer 60 which is a combined substrate comprising wound papers of different types. Desirable for the antistatic layer 60 considered as a barrier sheet or liner comprising 0.127 mm thickness with minimal impurities or additives generally a medical grade with a resistance classification (in Ohms) 1.0×10⁴ to <1.0×10¹¹ measured at 20-25% relative humidity by ANSI/ESD STM 11.11-2001 standards exhibiting a static dissipative property that does not readily attract or give up electrons. The antistatic layer 60 is listed centrally on the triboelectric series (see below) with static dissipative and innsulative properties that provide relatively little or no attraction for the drug clinging molecules of different aerosol formulas.

Other embodiments may include specialty papers and or materials and or treatments that enhance further the antistatic effects of the antistatic layer 60. Yet, spirally wound as a whole the core layer 56 and cover layer 54 for this embodiment have similar electrical and physical qualities as the antistatic layer 60 that consequently intensifies the antistatic attributes of the antistatic layer 60. The dielectric strength of a material is an intrinsic property of the bulk material and is dependent on the configuration of the material, which the field is applied. So in essence the whole paper tube body 22 is an antistatic spacer device.

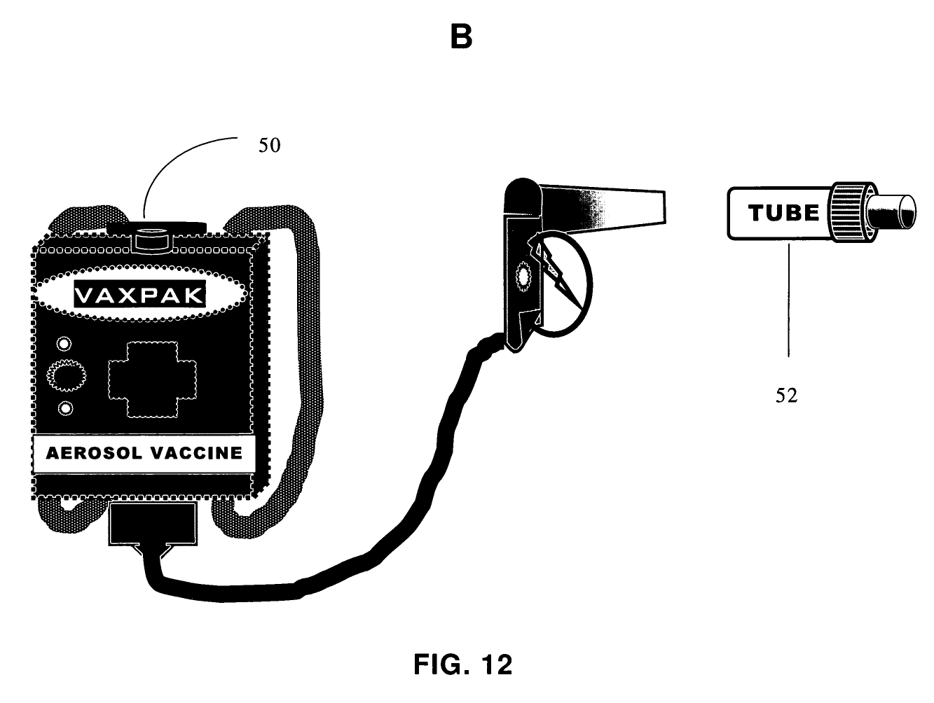

FIG. 12 displays a Mass Inoculation Pump (M.I.P.) 50 a disposable inoculator cartridge 52 a tube body 22 and replaceable mouthpiece 14 with a valve assembly. Other embodiments regarding the tube body 22 mouthpiece 14 and endcap 24 as a whole or in part FIGS. 9-10 include adaptations but not limited to: a Mass Inoculator Cartridge, a Lung Trainer, a Peak Flow Meter, a Muscle Exerciser, a Mucous Instigator.

The manner of using the disposable antistatic spacer to facilitate inhalation medicines is comparable to that for spacers in present use. But preferably, one first shakes rapidly their pMDI inhaler to mix medicine and propellant together and then inserts the pMDI assembly into the endcap 24 of the present invention. Next, after exhaling all air out of lungs, one should then hold the pMDI inhaler attached to the tube spacer 22 level and insert mouthpiece 14 into mouth, lastly while sipping in slowly one actuates the pMDI drum canister while continuing to sip in fully and then holds breath in for up to ten seconds allowing for the medicine to settle onto the lung tissue. A simple format to follow would be to instruct the patient to: Blow Out, Sip In, Press Down, Hold Breath and Count to Ten.

The embodiment seen in FIG. 12 of a tube cartridge 52 a replaceable mouthpiece and a disposable tube body is quickly attachable to a Mass Inoculation Pump (M.I.P.) 50 for efficient administration of large group or mass vaccinations. The M.I.P. can also be backpacked into third world countries for inoculation campaigns using the tube cartridge 52 as a needle free delivery device of life saving vaccines, just as well, schools and hospitals in need of a needle free device could benefit accordingly.

The manner of using the disposable antistatic spacer to facilitate other uses is by a simple in place modification and/or adding adaptations. As a low cost practice device the tube spacer can be used in schools, hospitals and clinics for educational and training use; modified with inexpensive digital electronics the tube platform can be used as a low cost disposable peak flow meter for asthma monitoring; with a spring tensioner the tubes paper core platform is an economical disposable muscle exerciser for post-op patients; adding a delay valve adaptation causing intermittent back pressure to the paper wound tube platform creates an environmentally friendly and much needed efficient mucous clearing device that would inexpensively relieve thousands of COPD patients.

Manufactured conveniently, the endcap, mouthpiece with or without a valve assembly may be constructed or formed totally with paper and or paper composites or by modular plastics or plastic composites or by rubber or rubber composites allowing for an economical disposable replaceable mouthpiece device. The paper tube body 22 may be constructed convulutly or spirally with the following processes.

In the papermaking process, wood is first chipped into small pieces. Then water and heat, and sometimes chemicals, are added to separate the wood into individual fibers. The fiber is mixed with lots of water, and then this pulp slurry is sprayed onto a huge flat wire screen that is moving very quickly through the paper machine. Water drains out, and the fibers bond together. The web of paper is pressed between rolls, which squeeze out more water and press it to make a smooth surface. Heated rollers then dry the paper, and the paper is slit into smaller rolls, and sometimes into sheets, and removed from the paper machine.

The paper tube body 22 could be wound anywhere in the world because of Marvin Stone. He patented the spiral winding process to manufacture the first paper drinking straws. Stone made his prototype straw by winding strips of paper around a pencil and gluing it together. He then experimented with paraffin-coated manila paper, so the straws would not become soggy while someone was drinking. The product was patented on January the 3, 1888.

In 1906, the first machine was invented by the “Stone Straw Corporation” to machine-wind straws, ending the hand-winding process. Later other kinds of spiral-wound paper and non-paper products were made. In 1928, electrical engineers began to use spiral-wound tubes in the first mass produced radios. All made by the same process invented by Stone. Spiral-wound tubing is now found everywhere: in electric motors, electrical apparatus, electronic devices, electronic components, aerospace, textile, automotive, fuses, batteries, transformers, pyrotechnics, medical packaging, product protection, and packaging applications.

Under the term tube winding, 3 ways of winding can be summarized: straight winding, conical winding and spiral winding. The process is as follows. First, ranging from 2 to max 30 plies are glued together, depending on the desired tube wall thickness and strength. These are spirally wound on a mandrel and fastened by means of 1 or 2 rubber belts. Thereafter the tube is cut to the desired length. The crush value (the force required to crush the tube) of the tubes is strongly influenced by the type of paper and the number of plies.

Tube winding adhesives/resins can be applied by means of rollers, a cascade system or a dipping tray. There are 2 types of adhesives: inner ply and outer ply adhesives. Some tube manufactures use the same glue for both the inner and outer plies. In this case, depending on the requirements and method of processing used by the manufacturer, a universal glue for both the plies can be offered. Typically the types of adhesives that can be used for tube winding include:

PVOH dispersion based, with or without filler;

Dextrin based in liquid or powder form (Dextrol)

Water glass based (sodium disilicate) either pure or modified;

PVA based powder (Kartofix)

Polyvinyl acetate (PVA) dispersions are fast setting and have a high solid content. PVOH (polyvinyl alcohol) dispersion adhesives often give a flexible adhesive film whereby a good sealing but a lower crush value is obtained. Dextrin adhesives have a high wet tack and a high solid content. The setting speed of these adhesives is in general slightly lower than that of PVOH dispersion adhesives. Dextrin adhesives give a hard adhesive film. Water glass (sodium disilicate) is not a true adhesive but more a product that fixates the plies in relation to each other. A high degree of sealing is not usually attained with water glass, however, high crush values are.

Tube winding adhesives must fulfill the following general properties for both inner and outer plies: ensure good adhesion of paper to paper (paper can have both a very open structure or a very closed structure); if the adhesive is applied to a single side, then there must be a good transfer to the dry paper plies (no excessive diffusion of the adhesive into the cardboard); to keep the moisture content of the tubes as low as possible, a high solid content is an advantage.

Paper swells under influence of moisture. It is therefore important to apply a minimal amount of water to keep the shrinkage of the tube to a minimum. With inner-ply adhesives if the adhesive is applied by means of submersion, then there must be low penetration into the paper. In outer-ply adhesives the adhesive must have a high wet tack or must set quickly; otherwise the tube ends will start to open upon cutting. Consideration of the electrical resistivities of the adhesives used may be important to pharmaceutical charge interactions but may also be non-substantial relative to the paper tube body 22 antistatic attributes especially if pure water based organic resins are used.

Issues of pharmaceutical electrostatic charge interaction to the functionality of therapeutic aerosols are well known. The electrostatic charge of inhalation medicines involves most aspects of their processing and general use. This includes their formulation, manufacture, dosing reproducibility and deposition behavior within the respiratory tract and within spacer devices.

Another significant electrostatic issue begins with the FDA announcement of a final rule to amend regulation 21 CFR 2.125 on the use of ozone-depleting substances (ODSs) based upon the Montreal Protocol in medical products. This rule establishes Dec. 31, 2008, as the date by which production and sale of single ingredient albuterol CFC pMDIs must stop. The Montreal Protocol is an important international environmental treaty banning substances that deplete the Ozone Layer to which the United States is a party.

Historically, pMDIs have used chlorofluorocarbon (CFC) propellants as the energy source needed to atomize the formulation into respirable drug particles. In regard to the use of CFCs in asthma aerosol inhalers, hydrofluoroalkanes (HFAs) are the preferred replacements because they contain neither chlorine nor bromine and therefore have no detrimental effect on stratospheric ozone. The most widely used HFA is HFA-134a, which has only one third of the ‘greenhouse’ (global warming) effect of the CFCs it replaces. Thus the conversion of industries from CFC use to HFA use has reduced and will reduce both stratospheric ozone depletion and global warming.

The reformulation of pMDIs with HFA-based propellants has resulted in changes to aerosol plume formation. Although this has implications for the design of add-on devices, smaller spacers or volume holding chambers (VHCs) 150 to 200 ml appear to be as effective as traditional large volume 750 mL devices. Unfortunately, many HFA formulations have significant electrostatic charges associated with their aerosols. This phenomena is produced upon actuation of the pMDI inhaler, making the development of spacers manufactured from charge dissipative materials timely. Due to the higher cost of the newly developed HFA pMDI formulas having affordable spacers with antistatic performance cannot be understated.

Fundamental to any discussion of the relevance of electrostatics in pharmaceutical aerosols is an understanding of the mechanism by which electrostatic charges develop. During aerosolisation processes for example shaking, priming, metering and dispersion, the relative movement of particles and droplets with inhaler device surfaces provides ideal conditions for the development of charge by frictional contact or “triboelectrification” whereby an electrostatic charge is generated by the contact and separation of two dissimilar materials, resulting in oppositely charged surfaces.

Understanding charge imbalances is quite easy. All matter is composed of atoms, whether the atoms are included in polymer molecules or in other arrangements. And all atoms are comprised of positively charged protons and neutrons in the nucleus (at the center) and “clouds” of negatively charged electrons around the nucleus in distinct layers (orbits). For a standard neutral atom, the number of electrons is the same as the number of protons. If an atom has more electrons than protons, it is negatively charged, and if it has more protons than electrons, then it is positively charged. The strength of the attachment of the electrons to the atom varies with the material some materials have their electrons more tightly bound to them than others.

The “triboelectric series” (below) is a rough measure of how tightly bound the electrons are in a given material. A material that is near the top of the series will, in general, give up electrons to become positively charged, and material that is near the bottom will, in general, capture electrons and become negatively charged, however, a material near the center will have a low propensity for charge.

Many related factors influence the mechanics of charge transfer and separation between dissimilar surfaces, including material properties (size, shape, surface roughness and purity, permitivity, frequency, duration and area of contact) and environmental conditions (temperature and relative humidity). Charge transfer in pharmaceutical aerosols is considered to be a function of both the formulation and the inhaler device components.

Triboelectrification in insulating materials such as pharmaceutical powders and inhaler devices is a complex surface phenomenon. While particle deposition by electrostatic charge in the respiratory tract is not considered to be a primary deposition mechanism, there is persuasive evidence that particle deposition in spacer devices is significantly influenced by electrostatics. Plastic spacers have an inherent electrostatic charge and because the aerosol cloud is confined inside the spacer, mutual repulsion between the charged particles causes them to move to the periphery of the aerosol cloud and connect with the spacer walls.

A simple example of aerosol medicines clinging to plastic spacer walls would be combing your hair on a dry day with a plastic comb. Human hair is significantly higher in the triboelectric series then plastic, so the hair will give up electrons to become positively charged, while the plastic comb will gain electrons to become negatively charged. Like charges repel one another, so the individual strands of hair will therefore repel one another. This results in a “bad hair day” due to a charge imbalance or as static electricity.

The charge transfer mechanism begins with the basic effect of adhesion. When two materials come into contact they form a bond, which may be very weak. If the materials are far apart on the triboelectric series, then the adhesion effect can lead to electron transfer. If the materials are separated, the charge imbalances remain and static electricity is created.

Triboelectric charge generation by materials is widely believed to be dependent on the surface resistivity of the materials in question. If a material has a low resistivity it is sometimes regarded as having a low propensity for charge generation. Surface resistivity and charge generation cannot be correlated. Still, the belief of a relation of these two parameters persists. For a material to be “antistatic” it must prevent or inhibit the buildup of static electricity.

The triboelectric series lists various materials according to their tendency to gain or lose electrons. It usually lists materials in order of decreasing tendency to charge positively (lose electrons), and increasing tendency to charge negatively (gain electrons). Important to antistatics is the middle of the list where materials that do not show strong tendency to behave either way.

THE TRIBOELECTRIC SERIES

| | |
|---|---|
| AIR | POSITIVE |
| HUMAN HANDS, SKIN | |
| ASBESTOS | +++ |
| RABBIT FUR | |
| GLASS | |
| HUMAN HAIR | ++ |
| MICA | |
| NYLON | |
| WOOL | |
| LEAD | + |
| CAT FUR | |
| SILK | |
| ALUMINUM | |
| PAPER | |

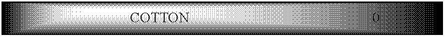

| | |
|---|---|
| STEEL | |
| WOOD | |
| LUCITE | |
| SEALING WAX | |
| AMBER | |
| RUBBER BALLOON | |
| HARD RUBBER | − |
| MYLAR | |
| NICKEL | |
| COPPER | |
| SILVER | |
| BRASS | |
| SYNTHETIC RUBBER | |
| GOLD, PLATINUM | |
| SULFUR | −− |
| ACETATE, RAYON | |
| POLYESTER | |
| CELLULOID | |
| POLYSTYRENE | |
| ORLON, ACRYLIC | |
| CELLOPHANE TAPE | |
| POLYVINYLIDENE CHLORIDE (SARAN) | |
| POLYURETHANE | |
| POLYETHYLENE | |
| POLYPROPYLENE | −−− |
| POLYVINYLCHLORIDE (VINYL) | |
| KEL-F (PCTFE) | NEGATIVE |
| SILICON | |
| TEFLON | |
| SILICONE RUBBER | |

Materials with surface resistivities in the static dissipative range will not retain static charges accumulated by tribocharging, especially if grounded. An apparatus designed together with appropriate static dissipative material and low tribo generating characteristic will offer the best “antistatic” protection.

Although complex antistatic chemical additives have been developed for plastic spacers the dissipative plastics and chemical technologies that achieve static dissipation are limited.

An amine is a long chain of carbon atoms surrounded by hydrogen atoms. This hydrocarbon chain is derived from a fatty acid and can vary from a few carbon atoms long to twenty or more carbon atoms. The last carbon is connected to a nitrogen atom, which has two hydrogen linked on the sides. Because simple fatty amines need a higher affinity for water to perform well as antistatic additives, ethylene oxide is reacted with them to make ethoxylated fatty amines.

This gives the molecules two polar end groups: OH or alcohol groups. Ethoxylated fatty amines are the additives used in dissipative polymer amine technology. Amide additives used in dissipative polymers are similarly based on a fatty acid attached to nitrogen that is also reacted with ethylene oxide. However, the carbon atom next to the nitrogen has a double bond attachment to oxygen instead of two single bonds to two hydrogen's. In this case, the molecule is specifically an ethoxylated fatty amide. It should be noted that the commercial nomenclature “amine free” usually means that an amide additive is used instead of an amine.

Other static dissipative additive systems for polyethylene exist with performance and properties very similar to amines and amides. Either type, the dissipative polymer starts as a homogeneous blend of additive, antiblock and polyolefin resin.

However, two things must occur for static dissipation to take place. The amine or amide must first diffuse through the plastic volume to reach and wet the surface. This is commonly referred to as blooming. Good compatibility between the additive molecule and the resin results in an insufficient amount of amine or amide wetting the surface. Without enough additive on the surface, static dissipation will not occur. Too little additive and resin compatibility results in too much additive getting to the surface. In this case, the static dissipative property will work well but the surface will be excessively greasy with the additive contaminating everything it contacts.

To work the additive must absorb at least a trace of atmospheric moisture with its two “claws.” This hydrogen-bonded combination of additive and water is the surface that provides static dissipation. Essentially, the conductivity is provided by the layer of water attached to the bonds and helped by the ionic solution of the additive. It is an ionic soup. But, during certain seasons in some locations, there is too little atmospheric moisture to be absorbed.

The term “compatibility” when used in conjunction with polycarbonates has a different meaning than that used to describe additive and resin compatibility. A more accurate term would probably be polycarbonate “coexistence.” Some antistatic additives such as tertiary amines cause polycarbonate parts to crack or “craze”. Leading to discoloring and decay of the polymers antistatic ability. Consequently, many dissipative polymers lose their electrical anti-static properties after some period of time at storage conditions, which is shortened by photo deteriation by the sun.

Just as important, the recycling nightmare associated with the many types of plastics used in spacer manufacture including: polyethylene terephthalate (PET), polypropylene (PP), polyethylene (LDPE) all need their own recycling process. Most plastics are derived from oil or gas, the extraction and processing of which requires large amounts of chemicals and, of course, generates waste (including hazardous waste).

Not only ecologically a better choice, choosing paper over plastic is a better natural antistat as well. Not only is paper a static dissipative material with low tribo generating characteristic papers natural dielectric strength is an efficient supporter of electrostatic fields.

When the flow of current between opposite electric charge poles is kept to a minimum and the electrostatic lines of flux are not impeded or interrupted, an electrostatic field can store energy. Papers dielectric ability supports an electrostatic field while dissipating minimal energy in the form of heat. The lower the dielectric loss (the proportion of energy lost as heat), the more effective is the dielectric material. Papers good dielectric constant of 3.7 is the extent to which it concentrates the electrostatic lines of flux. Materials with good dielectric constants like paper include ceramics, distilled water, mica, and glass.

The present “disposable antistatic tube” is an efficient dielectric material and electrical insulator, which keeps the electric field maintained with zero or near-zero power dissipation. This results in an almost no charge change or static cling of aerosol drug to its inner antistatic layer. The standard dielectric strength for paper is tested to be 16 MV/m refering to the maximum electric field strength that it can withstand intrinsically without breaking down or experiencing failure of its insulating properties. The theoretical dielectric strength of a material is an intrinsic property of the bulk material and is dependent on the configuration of the material which the field is applied

Many other types of paper tube materials available have even higher dielectric strengths. For instance the Precision Tube Company of Wheeling Ill. offers specialized papers convoluted or spiral wound that include: Dielectric Kraft, Vulcanized Fiber (fish paper), Thinwall (phenolic resin), Nomex, Kapton, Pyroform 310 and Flame Retardant Resinite for the electrical and electronic industries and could work well as an increased antistat for paper tube spacers.

Even coating applications for amplifying antistatic properties of paper could be considered that include a number of different techniques such as extrusion, dispersion coating, solution application and dry surface treatment (DST). These converting techniques, which in general are off-line processes, give vast possibilities to form coated, multilayer laminated paper tube structures. Yet simultaneously, the performance of the converting product must meet the requirements on cost-effectiveness during the manufacture and use, and more often, also during recycling and disposal. Therefore, often a compromise between the costs and the desired characteristics of the materials must be found.

When considering everything, a spiral wound paper tube will deliver more medicine to the intended site deeper inside the lungs then a conventional plastic spacer at less cost financially and environmentally. With modular or organic mouthpieces, end caps adaptable to standard and forth coming newer pressurized inhalant applicators; the present invention is an innovative device that accomplishes much for little.

The National Coalition on Health Care NCHC notes that health care spending continues to rise at the fastest rate in our history. In 2005, total national health expenditures rose 6.9 percent, which is two times the rate of inflation. Total spending was $2 TRILLION in 2005, or $6,700 per person. Total health care spending represents 16 percent of the gross domestic product (GDP). U.S. health care spending is expected to increase at similar levels for the next decade reaching $4 TRILLION in 2015, or 20 percent of the GDP.

Because “Second Generation” spacers are pricey and are not able to consistently deliver the full respirable dose, they have failed and are not popular with the public. To be popular, what is needed, is a “Next Generation” spacer that will in fact do the job as intended, be easier to use, does not waste medication and is offered at an affordable price.

From the descriptions above the advantages of the “disposable antistatic spacer” become more apparent:
(I) A few spiral winds of laminated medical grade paper provides a durable, crush resistant and moisture resilient disposable antistatic spacer “tube” that is more cost efficient to produce and less expensive to own.
(II) The disposable antistatic spacer does not need the extra priming puffs recommended for plastic spacers and will therefore not waste the new more costly HFA pMDI medication.
(III) No assembly is required with the disposable antistatic spacer and no maintenance cleaning is ever needed to maintain its function making the instructions less exhaustive and less complicated for patients.
(IV) The throwaway (whole or part) attributes of the disposable antistatic spacer, offers superior germ-free antibacterial operation in hospitals, schools or with mass inoculation campaigns.
(V) Offering the basic requirements needed for efficient operation the disposable antistatic spacer has sufficient length for proper medicine dispersion slowing and a one-way valve for coordinating patient use.
(VI) The disposable antistatic spacer's total dielectric properties deliver more medicine to the lungs without attracting drug molecules to its interior walls even after numerous uses.
(VII) With a 100% biodegradable core the disposable antistatic tube sustains resources, conserves manufacturing energy, and is less labor intensive or hazardous to recycle.
(VIII) The pioneering platform technology of the disposable antistatic tube spacer offers efficient in place adaptability to different pressure filled inhalant applicators and to future inhaler devices.
(IX) The chamber core of the disposable antistatic tube spacer with the replaceable mouthpiece is a revolutionary “needle free” answer for mass aerosol inoculation campaigns. This “inhalant tube cartridge” is an inexpensive spacer device for Mass Inoculation Pump applications against various biological and pandemic threats.

What is claimed is:

1. A disposable antistatic pulmonary therapeutic device comprising:
(a) a wound substrate defining a tubular body having generally a unrestricted flow therethrough comprising;
(b) an entrance inlet,
(c) an opposite exit outlet, wherein said tubular body has adhered on the inner surface thereof;
(d) at least one antistatic layer, wherein said antistatic layer comprises;
(e) a resistivity of 1×10¹³ Ohms/Sq or less.

2. The device of claim 1 wherein said wound substrate and said antistatic layer have a thickness difference of at least 0.05 mm.

3. The device of claim 1 wherein said antistatic layer has a neutral triboelectric effect such that it does not tend to readily attract or give up electrons when brought in contact or rubbed by hand by or with other materials.

4. The device of claim 1 wherein said antistatic layer is comprised by at least one member selected from the group consisting of paper, treated paper, paper composite, or applied coating.

5. The device of claim 1 wherein said tubular body is at least 100 mm in length and has at least a 25 mm inner diameter.

6. The device of claim 1 wherein said tubular body comprises oriented laminates.

7. The device of claim 6 wherein said oriented laminates are comprised by at least one member selected from the group consisting of gloss paper, matte paper, white paper, natural paper, color paper, printed paper, Kraft paper, medical grade paper, cotton paper, treated paper, or hybrid paper.

8. An antistatic pulmonary therapeutic device comprising:
(a) a wound substrate defining a tubular body having generally a unrestricted flow therethrough comprising;
(b) an entrance inlet,
(c) an opposite exit outlet wherein is deposited a mouthpiece, wherein said tubular body has adhered on the inner surface thereof
(d) at least one antistatic layer, wherein said antistatic layer has
(e) a resistivity of 1×10¹³ Ohms/Sq or less.

9. The device of claim 8 wherein said mouthpiece comprises at least one member selected from the group consisting of paper, cardboard, fiberboard, plastic, and paper/plastic hybrids.

10. The device of claim 8 wherein said mouthpiece comprises at least one member selected from the group consisting of white, color, clear, natural.

11. The device of claim 8 wherein said mouthpiece comprises a staged configuration with at least one stage.

12. The device of claim 11 wherein said mouthpiece comprises a body stage for receiving said tubular body and a lip stage.

13. The device of claim 12 wherein said body stage comprises
(a) a one-way valve, wherein said one-way valve comprises
(b) a valve seat,
(c) a valve seat stem,
(d) a valve diaphragm.

14. The device of claim 13 wherein said valve diaphragm comprises at least one member selected from the group consisting of paper, rubber, nitrile, plastic, and plastic/rubber hybrids.

15. The device of claim 13 wherein said one-way valve is sandwiched interiorly between said body stage and said tubular body.

16. The device of claim 8 wherein said entrance inlet is deposited an endcap.

17. The device of claim 16 wherein said endcap comprises a center orifice for receiving insertion of a medicinal inhalation applicator.

18. The device of claim 16 wherein said endcap comprises at least one member selected from the group consisting of paper, cardboard, fiberboard, plastic, nylon, rubber, nitrile and paper/plastic hybrids.

19. The device of claim 18 wherein said removable endcap comprises antiroll striations on the exterior sides and at least one member selected from the group consisting of white, color, clear, or natural.

20. A pulmonary drug delivery device comprising a hollow tubular substrate; wherein said hollow tubular substrate comprises a core of at least one member selected from the group consisting of; paper, cardboard, plastic, paper/plastic hybrid, blow-cell foam; wherein said core has adhered on the outer surface thereof at least one outer liner; wherein said core has adhered on the inner surface thereof at least one antistatic layer, wherein said antistatic layer comprises a resistivity of 1×10¹³ Ohms/Sq or less.
